(19) United States
(12) Patent Application Publication
(10) Pub. No.: US 2023/0044504 A1
(43) Pub. Date: Feb. 9, 2023

(54) MEDICAL APPLIANCE THAT CAN BE SET DOWN OR HUNG UP

(71) Applicant: AXESS VISION TECHNOLOGY, JOUE-LES-TOURS (FR)

(72) Inventor: Emmanuel HALLAUER, SACHE (FR)

(21) Appl. No.: 17/793,069

(22) PCT Filed: Dec. 22, 2020
(86) PCT No.: PCT/FR2020/052596

§ 371 Date: Jul. 15, 2022

(30) Foreign Application Priority Data
Jan. 17, 2020 (FR) FR2000449

(51) Int. Cl. A61B 1/00; H05K 5/02

(52) U.S. Cl. CPC A61B 1/00048; H05K 5/0234

(57) ABSTRACT

A medical appliance including a housing having a front face for displaying and, on the opposite side, a rear face, the housing being equipped with at least three legs to keep the medical appliance in a stable position when set down on a supporting surface, at least one of the legs being a hanging leg projecting out from the housing in the form of an arm and ending at its free end in a curved part, the housing being equipped with a fixing system for fixing to support equipment in such a way that each hanging leg is clear of the supporting surface so that at least one medical device can be hooked onto each hanging leg.

### TECHNICAL FIELD

The disclosure relates to the technical field of electronic medical appliances with display, intended to be moved during their use in order to be set down on a supporting surface or to be hung up on support equipment.

The subject matter of the disclosure finds a particularly advantageous application in the field where the electronic medical appliance is a monitor used with a medical device, such as an endoscope.

The technical field of medical endoscopes in the general sense makes it possible to access the inside of a hollow organ, a cavity or a natural artificial conduit of the human body, with a view to carrying out various operations for therapeutic, surgical or diagnostic purposes.

In the context of the disclosure, the endoscope can be used for diagnostic, therapeutic or surgical purposes in order to inspect all the internal parts of the human body that are accessible via the natural or artificial orifices. For example, the endoscope can be used in the field of urinary orifices, gastrointestinal orifices, the respiratory system, the cardiovascular system, the trachea, the sinus cavity, the female reproductive system, the abdominal cavity or any other part of the human body to be explored via a natural or artificial orifice.

In general, an endoscope includes a control handle in the form of a housing suitable for being held by a user of the endoscope and to which is fixed a tubular structure designed to be inserted in a body cavity of a patient to be examined. This tubular insertion structure includes a distal head equipped with a vision system for lighting and examining the organ, cavity or conduit of the human body. Upstream of the distal head, the tubular insertion structure includes a tip— deflection portion controlled by a mechanism of the control handle for orienting the distal head inside the insertion orifice. In order to visualise the interior of the hollow organ, cavity or natural or artificial conduit of the human body in which the endoscope is inserted, the endoscope is connected to a monitor on which the images coming from the vision system are displayed.

Fixed monitors are very common: this includes screens fixed to the wall of the operating theatre, for example. The stability of these screens is therefore guaranteed. However, in order to perform an endoscopy, it is then essential to be in a theatre equipped with a fixed monitor. In a hospital or a clinic, this requires either equipping all the theatres with monitors, or limiting the performance of endoscopy to certain theatres. This is why it is advantageous to be able to have a mobile monitor.

Mobile monitors are also known. These include free—standing screens, which can be set down on a supporting surface present in the operating theatre. Depending on the dimensions and weight of these screens, their movement is not always easy.

There exists, in particular, the mobile monitor marketed by AMBU® under the name AVIEW™, which is movable and small in size. This monitor has the advantage of not only being able to be set down on a supporting surface, but also being able to be fixed on an IV pole. To achieve this, the monitor includes, on its rear face, a U—shaped leg as well as a system for fixing to an IV pole. When the monitor is placed set down on a supporting surface, this is supported on the U—shaped leg. When the monitor is fixed on an IV pole, the U—shaped leg is folded away towards the screen in order to allow it to be fixed to the IV pole.

Furthermore, in the usual manner, the theatres where endoscopies are performed are equipped with cabinets provided with endoscope supports, as described in EP 1 290 983, or furniture with horizontal storage for endoscopes. In this case, the endoscopes are either stored in each theatre where an endoscopy is able to be performed, or stored in a room intended for storage where the user must go to find the endoscope before performing the endoscopy.

It is advantageous to have a mobile storage system which can be moved according to needs. Transport trolleys with bins are commonly used for this purpose. In addition to the size that such transport trolleys have, this solution has the disadvantage of having to move both the stock of endoscopes in the trolley and also the monitor used with such endoscopes.

Furthermore, a medical device is known from EP 3 229 898, which is provided with a display surface and intended to be moved. For this purpose, the described medical device includes support legs and a separate hook for a pivoting rail.

In this context, the applicant has highlighted the advantage of having a simple and space—saving solution for setting down and hanging up a medical display appliance while having nearby, in a limited space, the medical device associated with the appliance, such as an endoscope.

### SUMMARY

For this purpose, in the context of the disclosure, the medical appliance is mobile in order to be able to be set down on a supporting surface or hung up on support equipment, such as an IV pole or an IV stand, using a fixing system. The medical appliance also includes legs in order to keep it in a stable position when it is on the supporting surface. Advantageously, these legs do not need to be folded away when the medical appliance is fixed to the support equipment. Finally, the medical appliance is provided with at least one hanging leg allowing at least one medical device to be hooked, per hanging leg, and in particular at least one endoscope, to the medical appliance when this is fixed on the support equipment. Advantageously, this hanging leg does not need to be folded away when the medical appliance is set down on the supporting surface.

More precisely, the medical appliance according to the disclosure includes a housing having a front face for displaying and, on the opposite side, a rear face, the housing being equipped with at least three legs to keep the medical appliance in a stable position when set down on a supporting surface, at least one of the legs being a hanging leg projecting out from the housing in the form of an arm and ending at its free end in a curved part, the housing being equipped with a fixing system for fixing to support equipment in such a way that each hanging leg is clear of the supporting surface so that at least one medical device can be hooked onto each hanging leg.

The medical appliance according to the disclosure advantageously has one or the other of the following features, or a combination of two or more of the following features:— the housing includes two hanging legs projecting out from the housing in the form of an arm ending at its free end in a curved part;
the one or more hanging legs project out from the rear face of the housing;
each hanging leg includes an arm of length suitable to allow hooking of several medical devices one behind the other;
each hanging leg has a flat section;
the housing includes two support legs on the front face, and at least one hanging leg beyond the rear face;
the housing includes two support legs on the front face, which are formed by the lower ends of two gripping handles of the medical appliance extending on either side of the front face, projecting out from the housing;
at least one hanging leg is adjustable in inclination and/or in length.

The disclosure also concerns an equipment including a medical appliance as described within the scope of the disclosure, fixed to a support equipment by a fixing system. The medical equipment according to the disclosure enables the hooking of at least one medical device on at least one hanging leg of the medical appliance. Within the scope of the disclosure, the medical device is advantageously an endoscope in its sterile packaging.

Various other features arise from the description given below with reference to the appended drawings which show, by way of non—limiting examples, embodiments of the subject matter of the disclosure.

### BRIEF DESCRIPTION OF THE FIGURES

FIG. 1 is a view of the front face of a medical appliance embodied in the form of a monitor according to the disclosure.

FIG. 2 is a view of the rear face of a medical appliance according to the disclosure.

FIG. 3 is a 3/4 perspective view of the rear of a medical appliance according to the disclosure.

FIG. 4 is a profile view of a medical appliance according to the disclosure, placed on a supporting surface.

FIG. 5 is a profile view of a medical appliance according to the disclosure, fixed to support equipment.

### DETAILED DESCRIPTION

As illustrated in the figures, the subject matter of the disclosure relates to a medical appliance 1 enabling a display or visualisation of data or images. According to a preferred application, the medical appliance 1 according to the disclosure is a monitor. In the rest of the description, the medical appliance is also designated by monitor 1. Of course, this preferred example is not limiting.

The medical appliance for display or visualisation can be a medical monitor, for example.

The monitor 1 includes a housing 2 having a front face 2a and a rear face 2b extending substantially opposite the front face 2a, connected together by a peripheral region 3. The housing includes a lower edge 2c and an upper edge 2d on the opposite side, which are connected together by two lateral edges 2e and 2f. The peripheral region is then composed of the lower edge 2c, the upper edge 2d and the two lateral edges 2e and 2f.

The dimensions of the housing 2 are typically 300 mm in length, 200 mm in width, 110 mm in thickness. Hence, the monitor 1 is easy to transport due to its dimensions and its weight, while being intended to be set down on a supporting surface 4 or fixed to support equipment as will be explained in the rest of the description. Nevertheless, the dimensions of the housing 2 can vary without going beyond the scope of the disclosure, in the measure where the monitor 1 can be easily installed and moved from a supporting surface 4 to a support equipment and vice versa.

The housing 2 includes, on its front face 2a, a display surface 5 on which are displayed the images coming from a medical device to which the monitor 1 is connected. This display surface 5 can, for example, be an LCD screen or a touchscreen. According to the embodiment illustrated in the figures, the display surface 5 is smaller than the front face 2a of the housing 2. Alternatively, although not illustrated, the display surface 5 can completely cover the front face 2a of the housing 2, so that the front face 2a and the display surface 5 coincide.

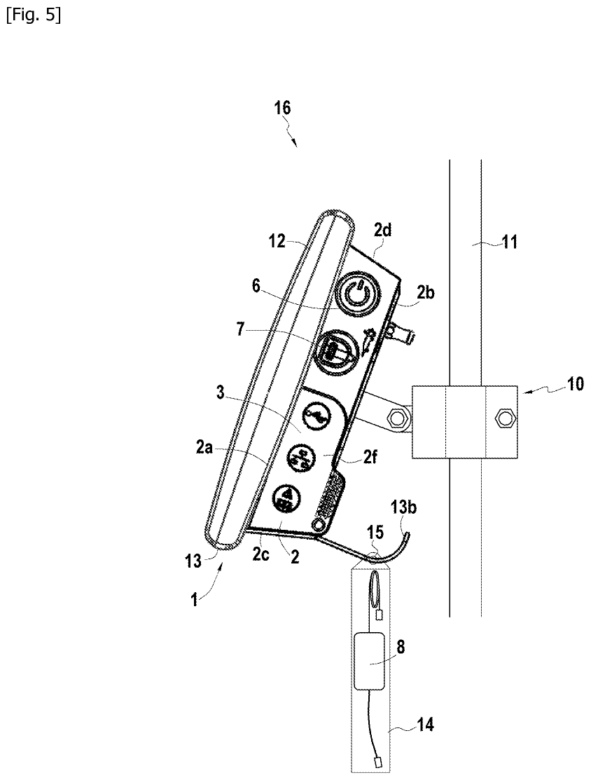

Control buttons 6 of the monitor 1 can be placed on the housing 2. These control buttons 6 make it possible, in particular, to switch on the display surface 5. In the exemplary embodiment illustrated in FIGS. 4 and 5, the control buttons 6 are located on the peripheral region 3 of the housing 2. This example is not limiting, and control buttons 6 can be placed either on the front face 2a, and/or on the rear face 2b and/or on the peripheral region 3 of the housing 2.

A first connector 7 for connecting the monitor 1 to a medical device 8 such as an endoscope can be present on the housing 2. Like the control buttons 6, this first connector 7 is located on the peripheral region 3 of the housing 2 in the example illustrated in FIGS. 4 and 5, but this example is not limiting and this first connector 7 could either be placed on the front face 2a, or on the rear face 2b or on the peripheral region 3 of the housing 2.

A second connector 9 for connecting the monitor 1 to the mains can also be present on the housing 2. Here too, this second connector 9 is located on the peripheral region 3 of the housing 2 in the example illustrated in FIGS. 4 and 5, but this example is not limiting and this second connector 9 could either be placed on the front face 2a, or on the rear face 2b or on the peripheral region 3 of the housing 2.

Other connectors can also be envisaged, such as USB ports or video outputs, for example, on the front face 2a, and/or on the rear face 2b and/or on the peripheral region 3 of the housing 2. Hence, a video output can be provided for an external monitor, a USB output for a remote control and/or an Ethernet output for connection to the computer system of a hospital centre for example.

The monitor 1 also has, on the rear face 2b, a fixing system 10 for fixing to support equipment 11. Preferably, the fixing system 10 enables a temporary or removable assembly of the monitor 1 with the support equipment 11.

Advantageously, the fixing system 10 is located substantially in the middle of the rear face 2b of the monitor 1.

According to an embodiment, the support equipment 11 includes a mast in the general sense, such as a tube, a rod, a column or a bar, for example. The mast forms part of the support equipment 11 of all known types, per se.

According to a preferred application, the support equipment 11 belongs to the medical field. By way of example, the support equipment 11 includes a fixed or mobile base from which the mast rises vertically. Advantageously, the support equipment 11 is mobile so that the base includes wheels.

The support equipment 11 can thus be a medical accessory, commonly referred to as an intravenous pole or an intravenous stand. Of course, the support equipment 11 can be produced in a different manner, such as in the form of an item of furniture or a trolley equipped with a mast extending over a more or less limited height.

In the example illustrated in the figures, the fixing system 10 is composed of a clamp in which a portion of the support equipment 11 can be inserted. The illustrated fixing system 10 thus makes it possible to place the monitor 1 on the support equipment 11 at the desired height. This example is not limiting, any fixing system known per se can be suitable within the scope of the disclosure.

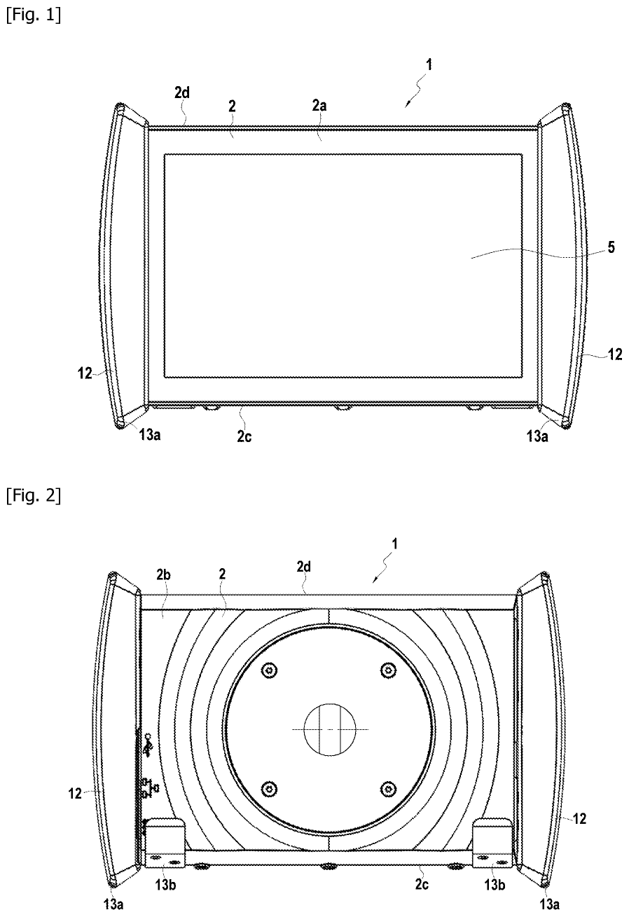

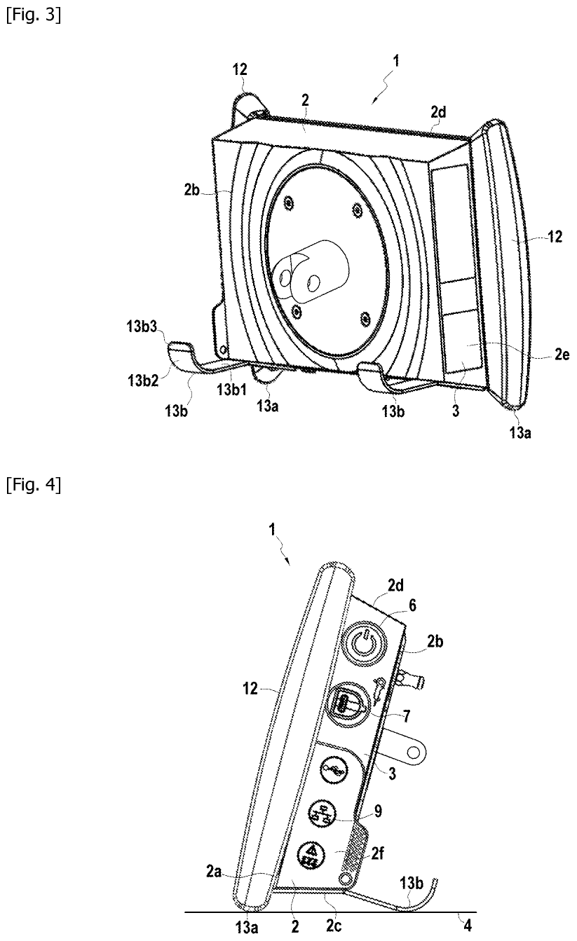

According to an embodiment of the disclosure, the monitor 1 has at least one gripping handle 12, and preferably two gripping handles 12 arranged on either side of the housing 2. In the exemplary embodiment illustrated in FIGS. 1 and 2 in particular, two gripping handles 12 extend on two opposite sides of the peripheral region 3 of the housing 2, and more precisely on the two lateral edges 2e and 2f. These gripping handles 12 project from the front face 2a of the housing 2. In the illustrated embodiment, the gripping handles 12 are trapezoidal—shaped with the free base curved. Any other shape which is easy to grip could nevertheless be envisaged within the scope of the disclosure. Advantageously, these gripping handles 12 protect the display surface 5, in particular for the case where the monitor 1 is set down supported by its front face 2a on a supporting surface 4.

In the context of the disclosure, the monitor 1 includes at least three legs 13a, 13b. These legs 13a, 13b are immobilised in order to form a support, in other words they are rigid. Advantageously, the legs 13a, 13b are not articulated. These legs 13a, 13b can project out from the housing 2 from its peripheral region 3, from its rear face 2b and/or from its front face 2a. Preferably, the legs 13a, 13b do not all project out from the same location on the monitor, in order to guarantee better stability of the monitor 1 when it is positioned on a supporting surface 4. Preferably, the legs 13a, 13b are arranged in at least two distinct planes and offset from the front towards the rear of the screen in order to guarantee good stability of the monitor 1 on the supporting surface 4.

The monitor 1 includes at least three legs 13a, 13b in order to ensure the stability of the monitor when this is placed on a supporting surface 4. However, the monitor 1 can include more legs 13a, 13b, and in particular four legs 13a, 13b. Of course, the end of the legs 13a, 13b are positioned in order to be situated in a same plane.

The legs 13a, 13b can have various shapes and dimensions, provided the monitor 1 can be set down stably and easily on the supporting surface 4. The position, shape and dimensions of the legs 13a, 13b are preferably such that they do not need to be folded away when the monitor 1 is fixed to the support equipment 11. In other words, the legs 13a, 13b are sufficiently far away from the fixing system 10 to allow an easy fixing to the support equipment 11, and with a geometry suitable for being clear of the support equipment 11 when the monitor is fixed thereto, for example by being offset with respect thereto. Thus, the monitor 1 according to the disclosure is easy to install and to move from a supporting surface 4 to the support equipment 11.

The legs 13a, 13b can be produced in various ways. These legs 13a, 13b can be assembled to the housing 2 being, for example, screwed, bonded or welded. These legs 13a, 13b can be integrated in the housing by being, for example, injected in a same part as the housing 2. In any event, the legs are assembled to the housing 2 in a rigid manner.

In the context of the disclosure, at least one of the legs projects out from the housing 2, in order to serve as a hanging leg 13b for at least one medical device 8. In addition, at least one of the legs 13a, 13b is not a hanging leg 13b so that such a leg, referred to as a support leg 13a, is used uniquely to be supported on the supporting surface 4.

Each support leg 13a can have any shape and is either placed on the front face 2a of the housing 2, on its peripheral region 3 or on its rear face 2b. Preferably, the legs 13a which are different from the hanging leg(s) 13b are placed on the front face 2a of the housing 2 or on its peripheral region 3.

A hanging leg 13b enables at least one medical device 8 to be hung when the monitor 1 is fixed to the support equipment 11. In a particularly advantageous manner, the medical device 8 hooked on the hanging leg 13b is an endoscope in its sterile packaging 14, as illustrated in FIG. 5. The sterile packaging 14 can include, at one of its ends, a loop or an orifice 15 in order to hook it onto the hanging leg 13b.

The one or more hanging legs 13b are either placed on the front face 2a of the housing 2, on its peripheral region 3 or on its rear face 2b. Preferably, as illustrated in the figures, the one or more hanging legs 13b project out from the rear face 2b of the housing 2.

The one or more hanging legs 13b are preferably arranged at a distance from the fixing system 10 in order to facilitate not only the fixing of the monitor 1 to the support equipment 11, but also the hooking on of the medical devices 8. In other words, the hanging leg 13b is sufficiently far away from the fixing system 10, and of suitable geometry, to be clear of the fixing system 10 and of the support equipment 11 when the monitor 1 is fixed thereto, so that it is easy to hook and remove the monitor 1 from the support equipment 11, and to hang, or remove, a medical device 8 when the monitor 1 is fixed to this support equipment 11.

The one or more hanging legs 13b are advantageously in the shape of a flat arm or blade with their free end being curved. The one or more hanging legs 13b have the shape of a straight arm 13b1 extending out from the housing and extending by a part 13b2 curved in an arc of a circle ending at a free end 13b3 oriented substantially perpendicular with respect to the arm. Advantageously, the straight arm 13b1 is rigidly extended by the curved part 13b2. The hanging leg 13b thus forms a hook for hooking the selected medical devices 8 in position hung by the curved part.

The length of the arm of the hanging leg 13b can be more or less large. The larger this is, the greater the number of medical devices 8 that can be hung one behind the other. Advantageously, the monitor 1 according to the disclosure allows hooking of several medical devices 8 one behind the other.

According to an embodiment of the disclosure, the hanging legs 13b are adjustable in length and/or in inclination. Thus, it is possible to adjust the angle of the monitor 1 when this is placed on the supporting surface 4.

The curved part of the hanging leg 13b is preferably oriented so as to hold at least one medical device 8 when this is hung. According to a particular embodiment, the curved part of the hanging leg has a vertical direction oriented towards the upper edge 2d of the housing 2.

According to an embodiment illustrated in the drawings, the one or more hanging legs 13b have a flat section. The sterile packaging 14 includes a loop or an orifice 15 of complementary shape to the flat section of the hanging legs 13b.

The one or more hanging legs 13b can of course be produced differently such as in the shape of a round rod.

Each hanging leg 13b must have a sufficient strength to enable hooking of at least one medical device 8, without risk of breakage or deterioration. Typically, the strength of each hanging leg 13b is at least 100 N.

In the exemplary embodiment illustrated in the figures, the monitor 1 includes four legs 13a, 13b: two support legs 13a projecting out from the front face 2a of the housing 2 and two hanging legs 13b projecting out from the rear face 2b of the housing 2. The two support legs 13a are positioned projecting out with respect to the lower edge 2c and substantially at the level of the two lateral edges 2e and 2f and are formed by the lower ends of the gripping handles 12. In this case, preferably at least the lower ends of the gripping handles 12 are made from or covered with an elastomer material in order to avoid any slipping on the supporting surface 4 and to improve the resistance to impacts. The two hanging legs 13b are positioned at the ends of the lower edge 2c of the housing 2, extending substantially perpendicular with respect to the rear face 2b of the housing 2. The two hanging legs 13b are thus located on either side of the middle of the rear face 2b of the housing in order to be positioned away from the fixing system 10. Hence, when the monitor 1 is assembled to the mast 3 using the fixing system 10, then the two hanging legs 13b are offset with respect to the mast, thus allowing the hooking on of medical devices 8.

The ends of the four legs 13a, 13b belong in a same plane so that the monitor 1 is stable when it is placed on a supporting surface 4, as illustrated in FIG. 4. The support legs 13a on the one hand and the hanging legs 13b on the other hand are aligned two by two, considering that the two hanging legs 13b are supported on the supporting surface beyond the rear face 2b of the housing 2. Good stability of the monitor 1 when set down on the supporting surface 4 is thus obtained.

When the monitor 1 is fixed to the support equipment 11, the two hanging legs 13b are clear of the supporting surface 4 thus allowing the hooking on of at least one medical device 8 and, in particular, the hooking on of an endoscope in its sterile packaging 14 through an orifice 15, as illustrated in FIG. 5. The medical devices 8 extend vertically in parallel with the mast and close to the mast of the support equipment 11. The medical devices 8 thus occupy a minimum of space while being close and easily accessible.

The disclosure also concerns an equipment 16 composed of the monitor 1 according to the disclosure, fixed to the support equipment 11 using the fixing system 10. At least one medical device 8 can be hung up on a hanging leg 13b, as illustrated in FIG. 5. According to the illustrated embodiment, the medical device 8 is an endoscope enclosed in a sterile packaging 14, and hooked onto a hanging leg 13b by an orifice 15. This equipment 16 includes all the elements necessary for performing an endoscopy for the case where the medical device 8 is an endoscope. This equipment 16 is mobile in the case where the support equipment 11 is provided with wheels.

The disclosure is not limited to the examples described and represented, because various modifications can be made thereto without departing from the scope of the disclosure.

1-11. (canceled)

12. A medical appliance comprising a housing having a front face configured for displaying and, on the opposite side, a rear face, the housing being equipped with at least three legs configured to keep the medical appliance in a stable position when set down on a supporting surface, at least one of the legs being a hanging leg projecting out from the housing in the form of an arm and ending at its free end in a curved part, the housing being equipped with a fixing system for fixing to support equipment in such a way that each hanging leg is clear of the supporting surface so that at least one medical device can be hooked onto each hanging leg.

13. The medical appliance according to claim 12, wherein the housing comprises two hanging legs projecting out from the housing in the form of an arm ending at its free end in a curved part.

14. The medical appliance according to claim 12, wherein the one or more hanging legs project out from the rear face of the housing.

15. The medical appliance according to claim 12, wherein each hanging leg has a flat section.

16. The medical appliance according to claim 12, wherein the housing comprises two support legs on the front face, and at least one hanging leg beyond the rear face.

17. A medical appliance according to claim 16, wherein the two support legs on the front face are formed by the lower ends of two gripping handles of said medical appliance extending on either side of the front face, projecting out from the housing.

18. A medical appliance according to claim 12, wherein at least one hanging leg is adjustable in one or more of inclination and length.

19. An equipment comprising a medical appliance according to claim 12, fixed to a support equipment by a fixing system.

20. The equipment according to claim 19, comprising at least one medical device hooked onto at least one hanging leg of the medical appliance.

21. The equipment according to claim 20, wherein the medical device is an endoscope in a sterile packaging.

22. The equipment according to claim 20, wherein the equipment comprises a plurality of medical devices hooked one behind the other onto at least one hanging leg.
